(19) United States
(12) Patent Application Publication
(10) Pub. No.: US 2007/0208277 A1
(43) Pub. Date: Sep. 6, 2007

(54) Vessel volume determination for embolization

(76) Inventors: Robert F. Rioux, Ashland, MA (US); Scott Sivak, Groton, MA (US)

Correspondence Address: FAY KAPLUN & MARCIN, LLP, 15O BROADWAY, SUITE 702, NEW YORK, NY, 10038, US

(21) Appl. No.: 11/369,451

(22) Filed: Mar. 6, 2006

(51) Int. Cl. A61B 5/103

(52) U.S. Cl. 600/587

(57) ABSTRACT

A system for vessel embolization, comprising a mapping element measuring dimensions of a target vessel and a computing element computing a volume of a selected portion of the target vessel based on the dimensions of the blood vessel in combination with an embolization material delivery element including a distal end which, when in an operative position, opens into the target vessel to deposit the embolization material into the selected portion of the target vessel.

### BRIEF DESCRIPTION OF THE DRAWINGS

FIG. 1 is a pictorial representation of a fluoroscopic localization of a blood vessels supplying a tumor;

FIG. 2 is a schematic representation of a probe mapping a blood vessel, according to the invention;

FIG. 3 is a diagram showing a mapped inner and outer wall of a blood vessel, according to the invention;

FIG. 4 is a diagram representing a blood vessel volume calculation according to the invention;

FIG. 5 is a table showing exemplary types of embolization elements used for embolization of a blood vessel according to the invention;

FIG. 6 is a schematic diagram showing delivery of embolization elements to a target blood vessel according to the invention;

FIG. 7 is a pictorial representation of a fluoroscopic evaluation of the embolization of a blood vessel; and

FIG. 8 is a diagram showing a flow sensing micro catheter according to the present invention.

### DETAILED DESCRIPTION

The present invention may be further understood with reference to the following description and the appended drawings, wherein like elements are referred to with the same reference numerals. The invention relates to methods and systems for treating tissue masses by reducing the flow of blood thereto. More specifically, the invention relates to a method and system for determining the volume of an embolization material for occluding a blood vessel supplying a target tissue mass such as a tumor.

Embodiments of the present invention determine the volume of a blood vessel or vessels supplying a target tissue mass to allow calculation of an optimum volume of embolization elements to be supplied thereto. The calculated amount of embolization elements is inserted into the vessel to occlude the vessel to necrose the target tissue while minimizing the impact on the surrounding non-targeted tissues. Additional steps according to the invention may be carried out to evaluate the effectiveness of the procedure by, for example, measuring the flow of blood through the occluded vessel after application of the embolization elements. Thus, the calculated amount of embolization elements and/or feedback from the measure of blood flow after the application may be used in selecting a size of embolization elements to be used and in determining when the embolization is completed.

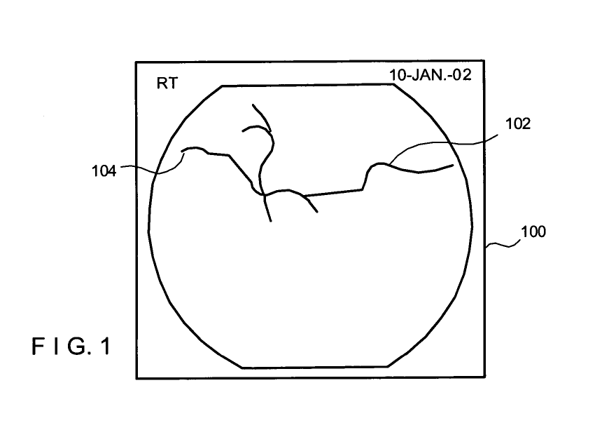

As shown in FIG. 1, the blood vessel or vessels that supply the target tissue mass are first identified. For example, a fluoroscopy screen 100 displays the target tissue mass (e.g., tumor 104) and the blood vessels 102 connected thereto. The physician may also use the image on the fluoroscopy screen 100 to determine an appropriate location for the deployment of embolization elements. In addition to fluoroscopy, other conventional methods of identifying and visualizing the target blood vessel supplying the tumor 104, such as a CT and contrast agent or MRI and contrast agent, may be used.

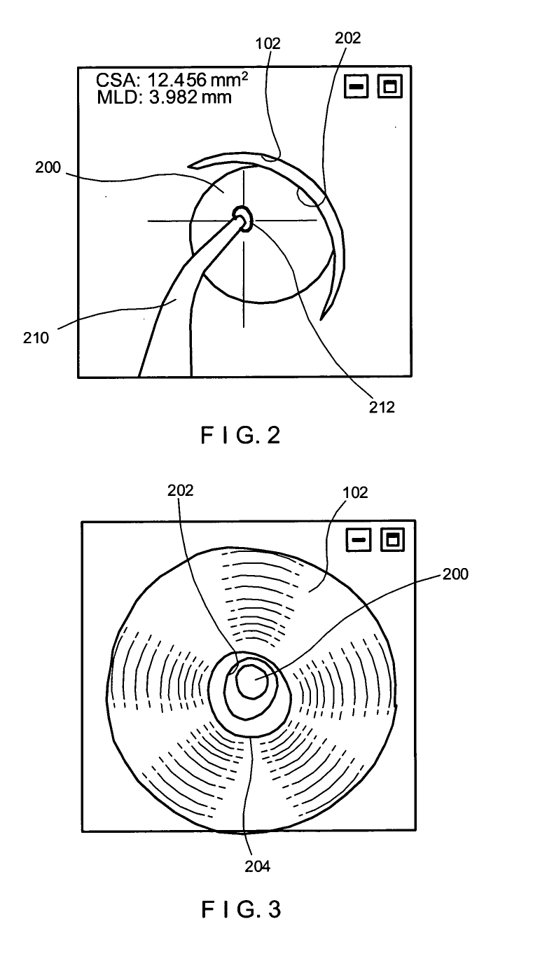

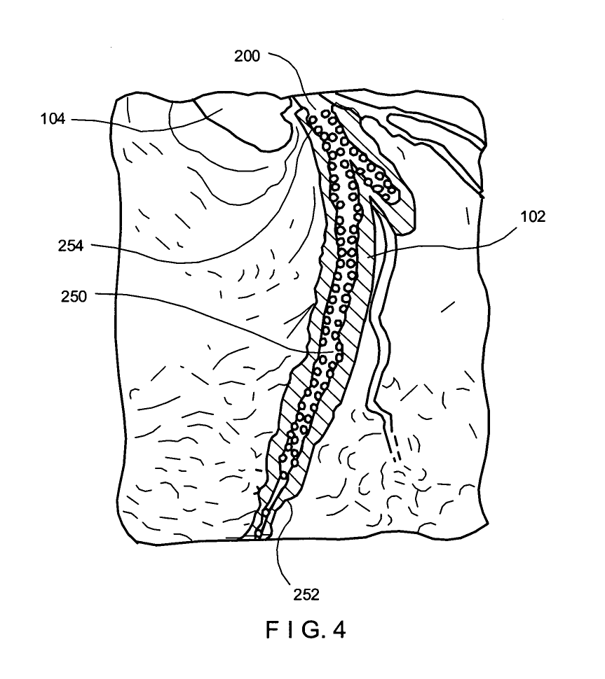

After identifying the target blood vessel(s), a visualization and mapping process according to the invention is carried out. The dimensions of the target blood vessel are determined by, for example, locating inner and outer walls at multiple locations along the length of each target vessel. Based on this data, volumes of target portions of each target vessel are calculated. FIGS. 2 and 4 show an exemplary embodiment of the procedure for mapping a target vessel 102. In this procedure, a probe 210 having a mapping head 212 is inserted into the target blood vessel 102 and translated along the length of a selected portion thereof. For example, the probe 210 may be a MediGuide visualization device, manufactured by MediGuide.

The visualization probe 210 is inserted into the lumen 200 of the target blood vessel 102 and advanced along the length of a selected portion of the lumen 200 in the vicinity of the tumor 104. As would be understood by those skilled in the art, the mapping head 212 is preferably adapted to identify the inner wall 202 and the outer wall 204 of the target blood vessel 102 to determine dimensions of the lumen 200. The apparatus according to the invention also tracks the location of the mapping head 212 within the blood vessel 102, so that a profile of the vessel along the longitudinal axis of the lumen 200 can be generated. As would be understood by those skilled in the art, the mapping head 212 may use any of a variety of known mechanisms to visualize and/or map the lumen 200. For example, ultrasound, CT, MRI, light or electrical energy may be used to measure dimensions of the target blood vessel 102, as well as other visual methods.

Embodiments of the invention utilize the dimensional data provided by the mapping head 212 of the probe 210 to compute an inner volume of the blood vessel 102. As would be understood by those skilled in the art, this volume from the measured data may be carried out using an electronic computer, a mechanical computer, numerical tables or graphs, or may be left for the physician to carry out as desired. In one exemplary embodiment shown in FIG. 4, the lumen 200 of the target blood vessel 102 is subdivided into a plurality of discrete sections 250 at each of which the probe 210 determines dimensions of the lumen by localizing the inner and outer walls of the vessel. The discrete sections 250 are located, for example, between a first point 252 distal of the tumor 104 and a second point 254 adjacent to the tumor 104, defining start/stop points for the volume computation.

According to the embodiments of the invention, the shape, size and volume of the vessel that is to be embolized is calculated based on the data provided by the probe 210 which may be, for example, a probe using MediGuide technology. Alternatively, a computing module may be provided that generates a value or other indication of the vessel's volume, using input generated by the probe 210. The computing module may display a 3-D image of the vessel, which would output the vessel volume in mL or CC. The computing module may directly provide to the physician the volume of the vessel, as well as an optimized amount and type of embolization elements for the procedure, based on input including dimensions of the vessel and a size and/or shape of the implants to be used, etc. Those skilled in the art will understand that a volume of the embolization elements to be introduced into the vessel will be different from a volume of a single embolization element multiplied by the number of elements to be provided as a packing efficiency of the elements will vary based on the size and shape of the elements. Thus, the volume calculated will preferably reflect a volume of a plurality of embolization elements packed as they will be in a target vessel.

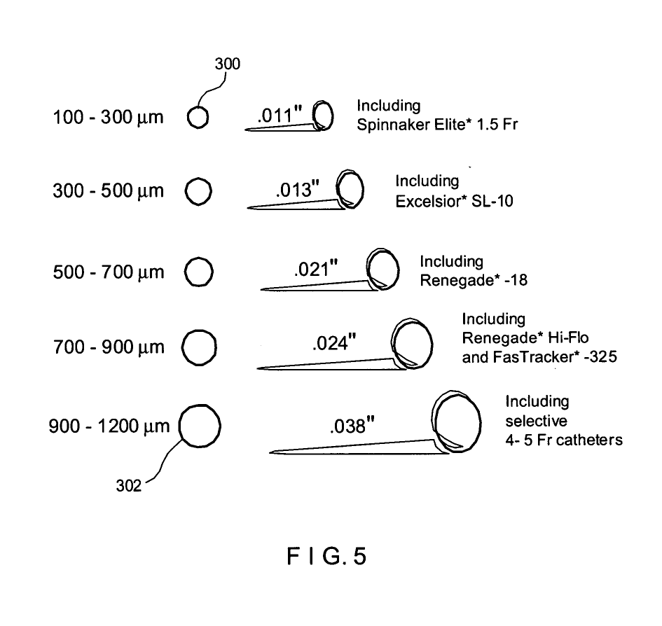

FIG. 5 shows a plurality of sample embolization elements which may, for example, be PVA spheres of different sizes which may be selected depending on the size of the target blood vessel(s). According to the invention, the correct size and number of embolization elements is determined based on the volume of the portion of the target blood vessel 102 as determined by the probe 210. For example, the exemplary embolization system according to the invention may comprise a table listing the dimensions of various embolization elements along with the volumes occupied by various numbers of the embolization elements.

Alternatively, the table of FIG. 5 may be included in an electronic module that provides the user with the appropriate size and an optimum number of embolization elements for occluding the target vessel 102, as derived from the data of the probe 210. In one exemplary embodiment, the embolization elements may range from small ellipsoids or spheres 300 having a diameter of about 100-300 μm, to large ellipsoids or spheres 302 having a diameter of about 900-1200 μm. These sizes of embolization elements are effective in occluding blood vessels having a lumen size of between about 1.5 Fr to about 4-5 Fr. Those skilled in the art will understand that, if a vessel smaller or larger than this range is to be occluded, embolization elements of larger or smaller size may be employed without using the same method to calculate the number/volume of these elements to be used.

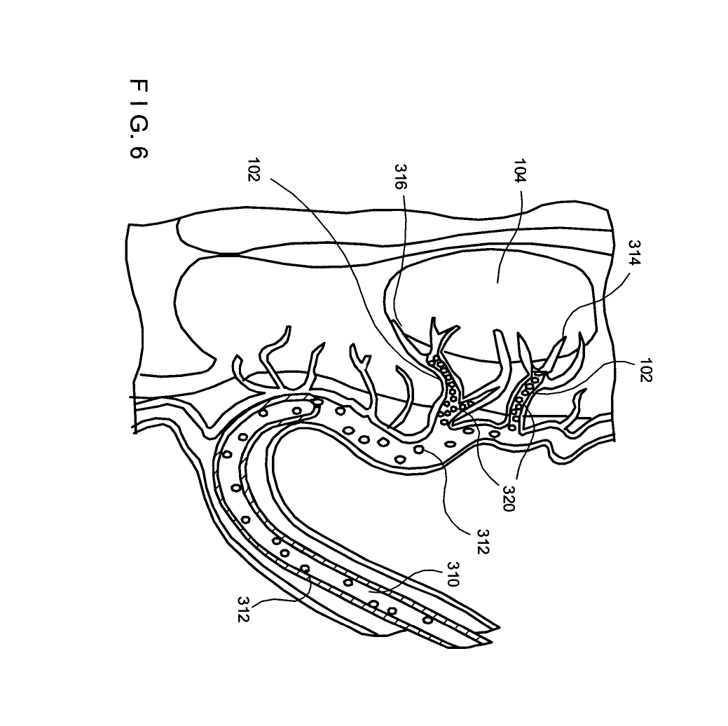

According to embodiments of the present invention, the appropriate number of embolization elements is injected into the target blood vessel to block the flow of blood therethrough. As shown in FIG. 6, an embolization delivery device 310 is advanced to the blood vessel or vessels 102 that feed blood to the tumor 104, so that the embolization elements 312 can be delivered thereto. For example, the embolization delivery device 310 may be a catheter or other similar device advanced along the vessel 102 until a target area is reached. When the delivery device 310 is in the target area, elements 312 or other embolization elements are introduced into the target vessel 102 by, for example, injecting the elements 312 into the delivery device 310 using a syringe or other injection mechanism. Once delivered, the elements 312 form an occlusion 320 in the blood vessel 102 preventing blood from reaching the ends 314, 316 of the vessel to necrose the tumor 104.

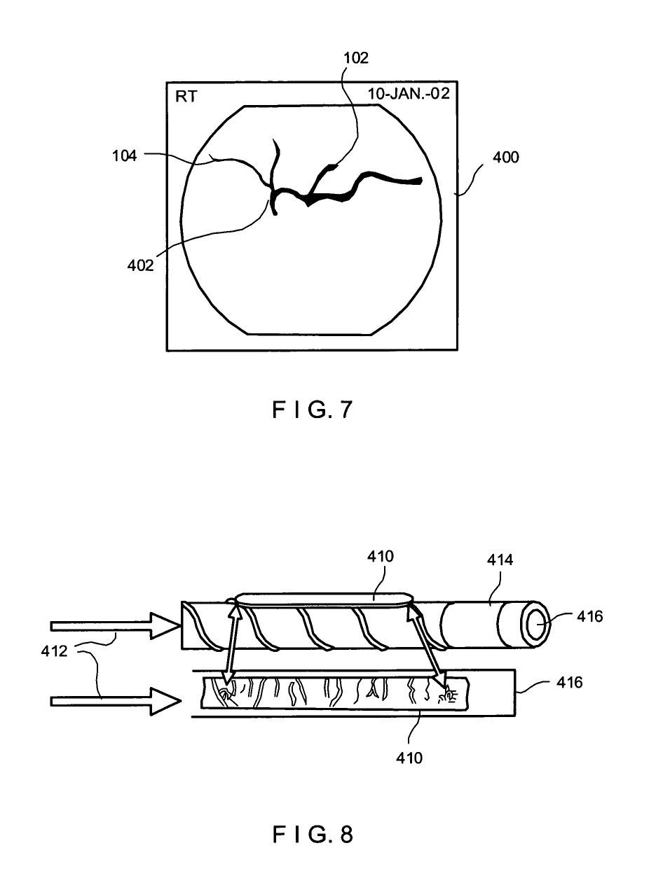

An evaluation step and system may be employed according to the invention to determine the effectiveness of the occlusion after the embolization elements have been delivered to the target blood vessel. For example, as shown in FIG. 7, a fluoroscopy display 400 may be aimed at the tumor 104 and target blood vessel 102 to determine the effectiveness of the occlusion 402. The shape and dimensions of the blood vessel 102, and in particular of the occluded region 402 may be evaluated to determine whether a sufficient number of embolization elements has been delivered to the target blood vessel 102. The tumor 104 may also be evaluated by fluoroscopy to determine whether a reduction in size occurs as a result of the treatment.

The effectiveness of the procedure according to the invention may also be evaluated with other diagnostic tools. For example, a flow sensing micro-catheter may be used to measure the flow of blood that passes across the occlusion, to determine the effectiveness of the embolization procedure. As shown in FIG. 8, the micro catheter 414 comprises a flow sensing element 410 that measures the flow rate of blood passing within the lumen 416 in the direction of the arrows 412. The flow sensing element 410 may comprise pressure sensors, electrostatic sensors, or other elements that can measure the flow of blood in the lumen 416. Signals generated by the flow sensing element 410 prior to the treatment of the target tissue mass are used to determine an initial state of the selected physiological condition. Signals generated after an embolic agent is dispensed to treat the tissue mass are used to determine the current state of the physiological condition. The initial and current states are compared to determine whether a desired change has been achieved. The flow sensing micro-catheter is referenced in application Ser. No. 10/739,584 filed Dec. 17, 2003.

The exemplary embodiments of the present invention provide an objective and efficient method of evaluating the type and amount of embolization material necessary to occlude a blood vessel supplying blood to a tumor. For example, the number and size of embolization elements used to occlude a blood vessel is derived from measurements of the internal dimensions of the blood vessel. The procedures and systems according to the present invention thus dispense with the trial and error approaches in which the physician guesses at the amount of material necessary for the embolization and after some time tests the results to determine if the amount was correct.

The present invention has been described with reference to specific embodiments, and more specifically to an embolization system using embolization elements such as PVA spheres. However, other embodiments may be devised that are applicable to other embolization materials, without departing from the scope of the invention. Accordingly, various modifications and changes may be made to the embodiments, without departing from the broadest spirit and scope of the present invention as set forth in the claims that follow. The specification and drawings are accordingly to be regarded in an illustrative rather than restrictive sense.

What is claimed is:

1. A system for vessel embolization, comprising;
a mapping element measuring dimensions of a target vessel;
a computing element computing a volume of a selected portion of the target vessel based on the dimensions of the blood vessel; and
an embolization material delivery element including a distal end which, when in an operative position, opens into the target vessel to deposit the embolization material into the selected portion of the target vessel.

2. The system according to claim 1, further comprising an evaluation element determining a post-procedure extent of embolization.

3. The system according to claim 1, further comprising an identification element visualizing the target vessel and a target tissue mass receiving fluid from the target vessel.

4. The system according to claim 1, wherein the mapping element comprises a probe insertable into the selected portion of the target vessel.

5. The system according to claim 4, wherein the probe comprises a mapping head measuring the selected portion of the target vessel with one of acoustic, optical and electric energy.

6. The system according to claim 1, wherein the embolization material comprises a plurality of embolization elements.

7. The system according to claim 2, wherein the volume computing element determines a desired size and number of embolization elements to be introduced into the target vessel.

8. The system according to claim 1, wherein the volume computing element comprises a correlation of embolization material and volume of the selected portion.

9. The system according to claim 1, wherein the volume computing element comprises a computer programmed to derive the volume of the target vessel from the dimensions measured by the mapping element.

10. The system according to claim 3, wherein the identification element includes a fluoroscopic visualization device.

11. The system according to claim 2, wherein the evaluation element includes one of a fluoroscopic visualization device and a flow sensing micro catheter.

12. A method of treating a target tissue mass, comprising:
identifying a target vessel supplying fluid to the target tissue mass;
mapping a portion of the target vessel near the target tissue mass;
determining an interior volume of the target vessel from data based on the mapping;
selecting a type and a volume of embolization material based on the interior volume determined; and
dispensing the embolization material into the target vessel to restrict the supply of fluid to the target tissue mass.

13. The method according to claim 12, further comprising evaluating a post procedure occlusion of the target vessel.

14. The method according to claim 12, further comprising mapping the target vessel with a probe having a mapping head locating inner and outer walls of the target vessel.

15. The method according to claim 12, further comprising mapping the target vessel to determine inner dimensions thereof.

16. The method according to claim 15, further comprising computing the interior volume of the target vessel from the mapped inner dimensions.

17. The method according to claim 12, wherein the embolization material includes a plurality of embolization elements and wherein selecting the type and volume of the embolization material includes selecting a diameter and a number of the embolization elements optimized to achieve a desired occlusion of the target vessel.

18. The method according to claim 12, further comprising dispensing the embolization material through a catheter.

19. The method according to claim 12, further comprising determining the interior volume of the target vessel by measuring an inner and an outer cross-sectional area of the target vessel at each of a plurality of locations along a longitudinal axis of the target vessel.

20. The method according to claim 19, wherein the locations are between a predefined start location adjacent the target tissue mass and a predefined end location distal of the start location.

21. The method according to claim 12, further comprising selecting the type and volume of embolization material using a computer based on the interior volume of the target vessel.
